(19) United States
(12) Patent Application Publication
(10) Pub. No.: US 2013/0197534 A1
(43) Pub. Date: Aug. 1, 2013

(54) APPARATUS AND METHOD FOR RESTRICTING MOVEMENT OF A CANNULA DURING A SURGICAL PROCEDURE

(71) Applicants: Alexander Lauderbaugh, Boulder, CO (US); Robert M. Scribner, Boulder, CO (US); Dan R. Hollowell, Longmont, CO (US); Richard W. Layne, Denver, CO (US); Robert T. Scribner, Lafayette, CO (US)

(72) Inventors: Alexander Lauderbaugh, Boulder, CO (US); Robert M. Scribner, Boulder, CO (US); Dan R. Hollowell, Longmont, CO (US); Richard W. Layne, Denver, CO (US); Robert T. Scribner, Lafayette, CO (US)

(73) Assignee: Crosstrees Medical, Inc., Boulder, CO (US)

(21) Appl. No.: 13/567,594

(22) Filed: Aug. 6, 2012

Related U.S. Application Data
(60) Provisional application No. 61/515,139, filed on Aug. 4, 2011.

(51) Int. Cl. A61B 19/00

(52) U.S. Cl. CPC A61B 19/00
USPC 606/108

(57) ABSTRACT

An apparatus comprises a body portion having a sidewall with one or more threads and defining a lumen. The apparatus includes a brace portion having a sidewall that defines a lumen that is fluidically coupled to the lumen of the body portion. The sidewall of the brace portion has one or more threads that are configured to matingly couple to the threads of the body portion. The brace portion has a contact surface configured to contact a bodily tissue of a patient when a cannula is disposed within the lumen of the body portion and the lumen of the brace portion. The brace portion and the body portion are collectively configured to maintain a position of the cannula within a body of the patient relative to the bodily tissue when the cannula is disposed within the lumen of the brace portion and the lumen of the body portion.

### REFERENCE TO RELATED APPLICATION

This application claims priority to and the benefit of U.S. Patent Provisional Application No. 61/515,139, filed Aug. 4, 2011, entitled “Apparatus and Method for Restricting Movement of a Cannula during a Surgical Procedure,” which is incorporated herein by reference in its entirety.

### BACKGROUND

The invention relates generally to medical devices, and more particularly to methods and apparatus for restricting the movement of a cannula during a surgical procedure.

Minimally invasive or percutaneous surgical procedures often involve the use of catheters and/or cannulas for providing access to internal tissue (e.g., bone, soft tissue, etc.). In such procedures, an opening is formed in the skin, either by making an incision or using a device such as a trocar. Once the opening is formed in the skin, the catheter or cannula can be advanced to a desired location. In connection with certain procedures, the cannula can be advanced into soft tissue and/or a bony structure.

In some surgical procedures, temporary or permanent implants are delivered through a cannula and positioned in a desired location. In some procedures, medical devices, or portions thereof, are removed through the cannula after a procedure is performed. In certain instances, when the force required to remove the medical device through the cannula is greater than the frictional force maintaining the position of the cannula, the cannula can be advanced further into the body, causing tissue damage or inhibiting the removal of the medical device from the body through the cannula. For example, in connection with a spinal procedure, a pod is delivered into a vertebra, filled with a bone cement and then extracted. When the pod is extracted through the cannula, the cannula may have enough frictional force from contact with the bone to prevent movement of the cannula. If the force retaining the pod in the vertebral body is greater than the cannula-bone interface frictional force, however, the extraction of the pod will force the cannula further into the body (i.e., in a distal direction) instead of pulling the pod out of the body (i.e., in a proximal direction).

Thus, a need exists for methods and apparatus for restricting movement of a cannula during a surgical procedure.

### SUMMARY OF THE INVENTION

In some embodiments, an apparatus includes a first portion and a second portion. The first portion includes a guide member and a coupling portion that collectively define a lumen configured to receive a cannula therethrough. The guide member is configured to move relative to the coupling portion between a first position and a second position when the cannula is disposed within the lumen. The first portion has a first configuration when the guide member is in the first position relative to the coupling portion. The first portion has a second configuration when the guide member is in the second position relative to the coupling portion. The second portion is coupled to a distal end of the first portion. The second portion defining a lumen configured to receive at least a portion of the cannula. The lumen of the second portion is in fluid communication with the lumen of the first portion. The second portion includes a contact surface that is configured to contact a bodily tissue of a patient when at least a portion of the cannula is disposed through the lumen of the second portion and within the body of the patient. The second portion and the first portion are collectively configured to maintain a position of the cannula relative to the bodily tissue when at least a portion of the cannula is disposed through the lumen of the first portion, the lumen of the second portion and within the body of the patient.

### BRIEF DESCRIPTION OF THE DRAWINGS

FIG. 1 is a schematic illustration of a cannula holder according to an embodiment of the invention.

FIGS. 2-8 are perspective views of apparatus according to embodiments of the invention.

### DETAILED DESCRIPTION

Medical devices are described herein. In some embodiments, a medical device includes a retainer having a brace and a body portion. The brace and body portion collectively define an opening configured to receive a cannula therethrough. The brace is configured to be disposed adjacent bodily tissue and the body portion is configured to be disposed between a hub of a cannula and the brace. The body portion is adjustable between multiple configurations such that it is configured to maintain the cannula in position relative to bodily tissue. As described in greater detail herein, in some embodiments, the brace is positioned against a patient's skin. In some embodiments, the brace is positioned against a bony structure. Accordingly, instead of relying completely on the friction between the cannula and the bone to prevent anterior (i.e., distal) advancement of the cannula, the load applied during removal of a medical device through the cannula is distributed onto other tissues. By increasing the area to which the withdrawal forces are applied, the cannula retainer reduces the probability of cannula advancement and associated negative clinical outcomes (e.g., tissue damage, incorrect device placement, etc.).

In some embodiments, the retainer is a modification of the cannula shaft, a modification to the cannula hub or a separate device that is attached to the cannula. The retainer can be permanently attached to the cannula, configured to slidably engage the cannula before placement into the patient, or configured to fit onto the cannula shaft in such a way as to allow installation after the cannula has been positioned in the patient.

In some embodiments, the cannula tip can be modified to include flutes, knurls, threads, etc to increase or supplement frictional forces between the cannula shaft and the bone. Such a modification can be instead of or in addition to use of the retainer as described herein. In other embodiments, the cannula can include a retractable retention mechanism configured to engage the bone to limit movement of the cannula shaft with respect to the bone.

In some embodiments, a method includes forming an opening in a bodily tissue, inserting a cannula into the opening, coupling a retainer to the cannula, and adjusting the retainer to maintain the position of the cannula relative to the bodily tissue. In some embodiments, the cannula is coupled to the retainer before the cannula is inserted in the bodily tissue.

As used in this specification, the words “proximal” and “distal” refer to direction closer to and away from, respectively, an operator (e.g., surgeon, physician, nurse, technician, etc.) who would insert the medical device into the patient, with the tip-end (i.e., distal end) of the device inserted inside a patient's body first. Thus, for example, the end of a medical device first inserted or closest to the inside of the patient's body would be the distal end, while the end of the medical device to last enter the patient's body or furthest from the body would be the proximal end of the medical device.

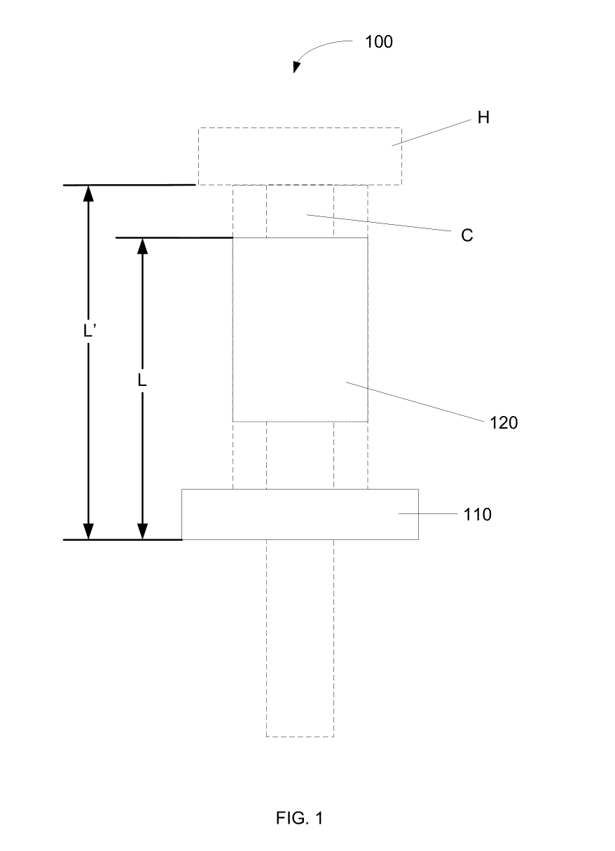

FIG. 1 is a schematic illustration of a retainer 100 according to an embodiment of the invention. As described herein, in some embodiments, the retainer 100 can be used to maintain a cannula or catheter in a location within bodily tissue. For example, the retainer 100 can be used to maintain the position of a cannula relative to a bone (e.g., a long bone, a vertebral body or the like), an organ, or soft tissue. Although described herein as being configured for use with surgical procedures involving vertebra, in other embodiments, the retainer 100 can be used with any percutaneous or minimally invasive procedure in which it is desirable to maintain the position of the access cannula or catheter.

The retainer 100 includes a brace 110 and a body portion 120. In some embodiments, the brace 110 is configured to be positioned against the skin of a patient. In other embodiments, the brace 110 is configured to be positioned against internal bodily tissue (e.g., a bone) into which the cannula is inserted. In embodiments in which the brace 110 is to be positioned against the bone, the diameter of the brace need only be slightly greater than the diameter of the opening in the bone through which the cannula is inserted.

The body portion 120 can be reconfigured or adjusted to facilitate maintaining the position of the cannula. In some embodiments, a length of the body portion 120 can be changed to prevent advancement of the cannula further into the body. For example, in a first configuration, the body portion has a length L. The body portion 120 is maintained in the first configuration until the cannula is positioned appropriately within the bodily tissue. Once the cannula is inserted to a desired depth/position, the body portion 120 is moved to a second configuration having a length L′, which is at least substantially equal to the distance between the hub H of the cannula C and brace 110.

As discussed in greater detail herein, in some embodiments, the body portion has multiple portions movable relative to one another such that the length of the body portion can change. For example, the body portion 120 can include a first portion and a second portion that are telescopically arranged, threadedly engaged, etc. such that the length L of the body portion can move to length L′.

In some embodiments, prior to insertion of the cannula, the brace 110 is positioned adjacent the body portion 120. After the cannula is inserted into the body and is appropriately positioned, the brace 120 is moved into position (i.e., against the skin or bone).

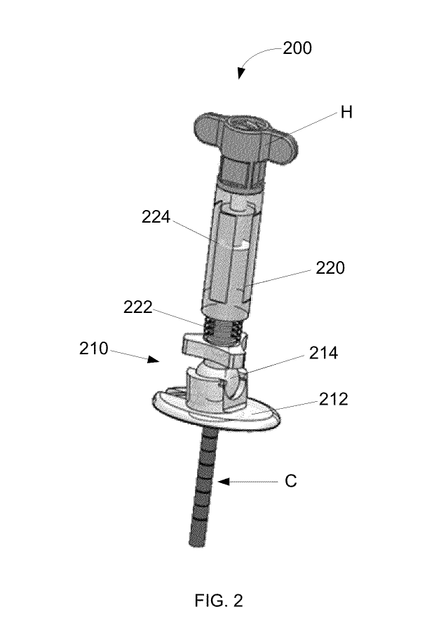

FIG. 2 illustrates a retainer 200 including a brace 210 and a body portion 220. The brace 210 and the body portion 220 are configured to receive a cannula C. The brace 210 includes a tissue-contacting portion 212 and a ball and socket joint 214. After the cannula is placed at the desired depth, the tissue-contacting portion 212 is extended to press against the patient's skin. The angle of the brace 210 is adjustable to account for different anatomies by using the ball and socket joint 214. In some embodiments, the brace is adjustable by a pin joint, swivel joint, or some other method.

The body portion 220 is adjustable by moving threaded adjustment portion 222 relative to barrel 224. As the barrel moves along the length of the threaded adjustment portion 222, the overall length of body portion 220 changes. In some embodiments, instead of using the threaded adjustment portion 222 and barrel 224, the body portion can include telescoping portions that are locked in place using friction fit, screws, pins, etc.

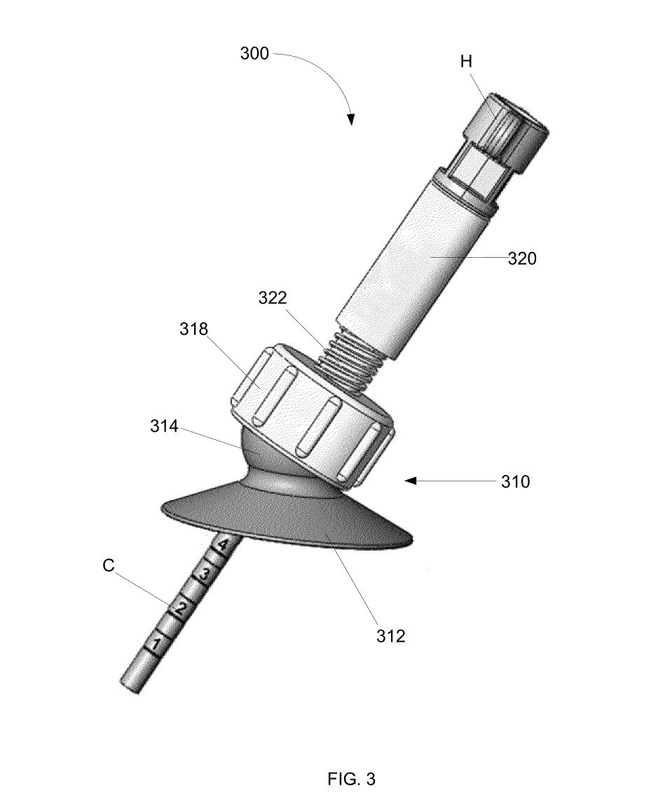

FIG. 3 illustrates a retainer 300 including a brace 310 and body portion 320. The brace 310 includes a skin contacting portion 312 and ball and socket joint 314. Body portion 320 includes a threaded post 322 disposed in socket 318 about which a barrel 324 is threadedly engaged. The distance between cannula hub H and brace 310 can be modified to maintain the cannula C in position relative to the tissue into which the cannula C is inserted.

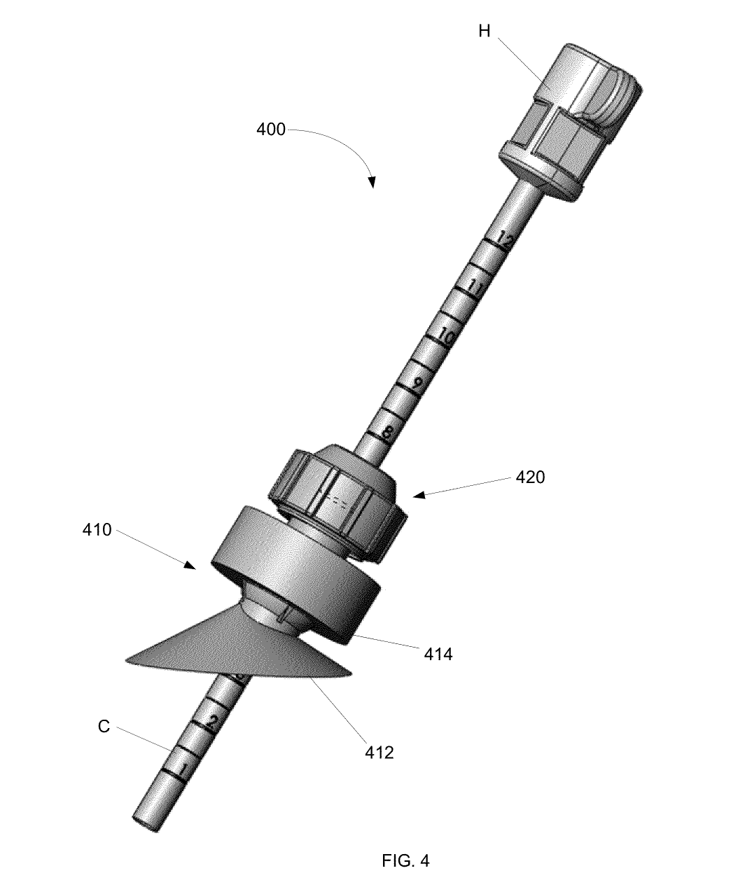

FIG. 4 illustrates a retainer 400 including a brace 410 and a body portion 420. Brace 410 includes a skin-contacting portion 412 and a ball and socket joint 414. The body portion 420 includes a collet locking assembly (not shown) configured to maintain the cannula C in place such that it cannot move relative to the brace 410. Body portion 420 is configured to be rotated from a first configuration in which the cannula C is free to slide through the retainer 400 and a second configuration in which the body portion 420 is locked around the cannula C, thereby preventing movement of the cannula C.

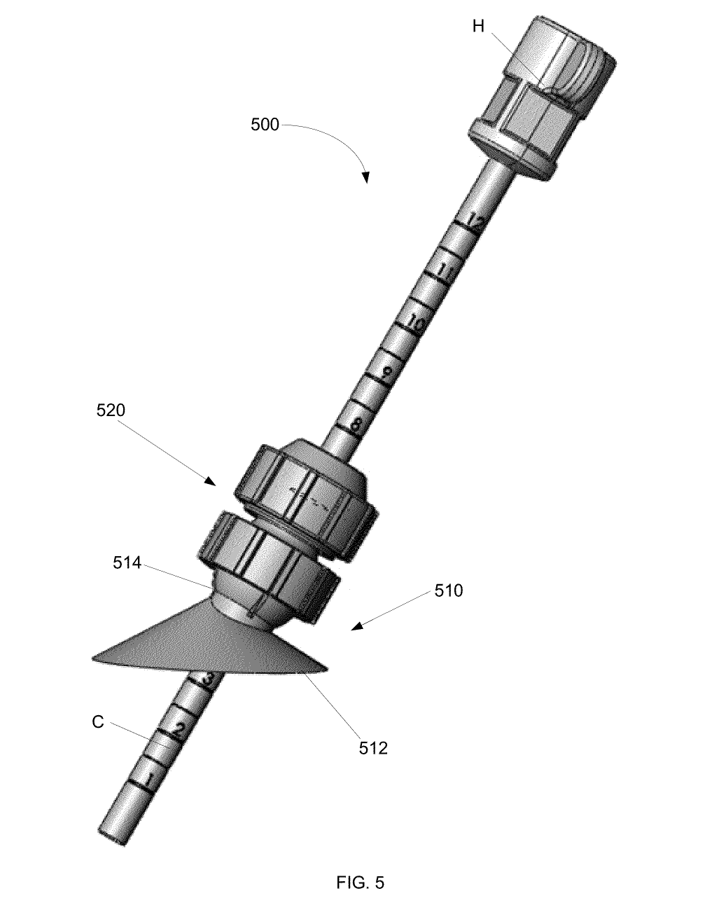

FIG. 5 illustrates a retainer 500 including a brace 510 and a body portion 520. Brace 510 includes a skin-contacting portion 512 and a ball and socket joint 514. The ball and socket joint 514 is configured to adjustably lock in place such that when a desired angle/position of insertion of the cannula C is achieved, the ball and socket joint 514 is prevented from further movement. The body portion 520 includes a collet locking assembly (not shown) configured to maintain the cannula C in place such that it cannot move relative to the brace 510.

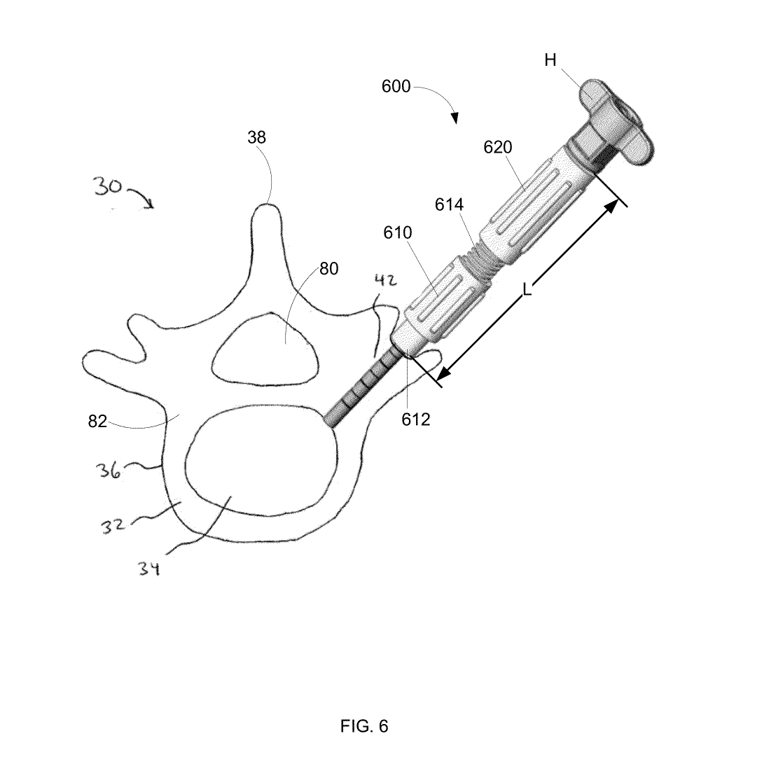

Although the retainer 100 shown and described above includes a brace 110 configured to be positioned against the skin of a patient, in other embodiments, a retainer can include a brace that can be at least partially disposed within the body and/or that can be positioned against the internal bodily tissue (e.g., a bone) into which the cannula is inserted. For example, FIG. 6 shows a retainer 600 that can be used to limit the movement of a cannula C relative to the internal bodily tissue into which the distal portion of the cannula C is inserted. More particularly, as described herein, the retainer 600 is configured to limit movement of the cannula C relative to the internal bodily tissue in the distal direction. In other embodiments, the retainer 600 can be configured to limit the movement of the cannula C in both the distal and proximal direction. In yet other embodiments, the retainer 600 can be configured to limit the rotational movement of the cannula C relative to the internal bodily tissue.

The internal bodily tissue can be any suitable bodily tissue within which a portion of the cannula C is disposed. For example, as shown in FIG. 6, the internal bodily tissue can be a vertebra 30. The vertebra 30 includes a spinous process 38, a spinal cord canal 80, a pedicle 82 and a vertebral body 36. The vertebral body 36 includes an outer portion 32 formed from a cortical bone surrounding an inner portion 34 formed from cancellous bone. As described herein, the distal portion of the cannula C can be disposed within a cavity defined within the inner portion 34 of the vertebral body 36. A pod and/or expandable member can be delivered into the vertebra 30, filled with a bone cement and then extracted.

The retainer 600 includes a brace 610 and a body 620. The brace 610 includes a contact surface 612 and a threaded portion 614. The body 620 includes a threaded portion that corresponds to the threaded portion 614 of the brace 610. In use, the brace 610 and the body 620 are each disposed about the shaft of the cannula C such that the proximal end portion of the body 620 is in contact with the hub H of the cannula, and the brace 610 is threadably coupled to the distal end portion of the body 620. As described above, the brace 610 can be rotated relative to the body 620 and/or the cannula C to adjust the length L between the contact surface 612 of the brace 610 and the hub H of the cannula C. Similarly stated, the brace 610 can be rotated relative to the body 620 and/or the cannula C to adjust the length L of the retainer 600.

In use, the retainer 600 is disposed about the cannula C, and the cannula C is inserted into the body such that a distal end portion of the cannula is within the vertebra and the contact surface 612 is disposed against the outer portion 32 of the vertebra 30. The distance within which the distal end portion of the cannula C is disposed within the vertebra 30 can be adjusted by adjusting the length L of the retainer 600, as described above. In some embodiments, the retainer 600 can be disposed into the body until the contact surface 612 is in position against the bodily tissue, and then the cannula C can be inserted into the body through the retainer 600 until the hub H contacts the proximal portion of the body 620 of the retainer 600.

Although shown in FIG. 6 as being disposed against the pedicle 42, in other embodiments, the contact surface 612 of the retainer 600 can be disposed against any suitable surface of the vertebra 30. In some embodiments, the contact surface 612 can have a shape and/or contour that corresponds to a contour of a portion of the bodily tissue against which the contact surface 612 is placed. In this manner, the contact surface 612 can be configured to evenly distribute any forces transmitted to the surface of the bodily tissue during the extraction process. In other embodiments, the contact surface and/or any other portion of any of the retainers disclosed herein can include any suitable mechanism and/or structure to increase the friction between the retainer and the bodily tissue against which the retainer is disposed. For example, in some embodiments the brace of any of the retainers described herein can include a surface feature, such as, for example, flutes, knurls, and/or threads, that is configured to engage a portion of the bodily tissue to limit movement of the retainer (and thus, the cannula) relative to the bodily tissue. In other embodiments, the retainer can include a movable and/or expandable portion configured to expand and/or move to engage a portion of the bodily tissue after the retainer is disposed with the body.

The size of the distal end portion of the brace 610 (i.e., the portion adjacent the contact surface 612) need only be slightly greater than the size of the opening through the outer portion of the vertebra 30. In this manner, the brace 610 is configured to be disposed into the body via the same incision and/or path through which the cannula C is disposed.

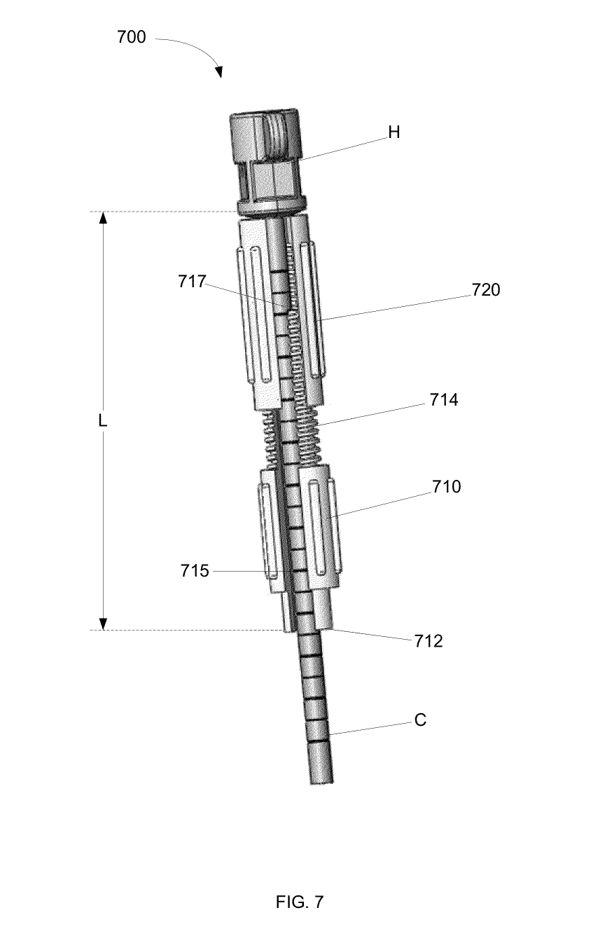

Although the retainer 600 is shown and described as circumscribing the cannula C, in other embodiments, a retainer need only surround a portion of the cannula C. In such embodiments, the retainer can be disposed about the cannula after the cannula has been inserted into the body. For example, FIG. 7 shows a retainer 700 according to an embodiment. The retainer 700 includes a brace 710 and a body 720. The brace 710 includes a contact surface 712 and a threaded portion 714. The brace 710 defines an opening 715 such that the brace 710 only partially circumscribes the cannula C.

The body 720 includes a threaded portion that corresponds to the threaded portion 714 of the brace 710. The body 720 defines an opening 717 such that the body 720 only partially circumscribes the cannula C. In use, the brace 710 and the body 720 are each disposed about the shaft of the cannula C such that the proximal end portion of the body 720 is in contact with the hub H of the cannula, and the brace 710 is threadably coupled to the distal end portion of the body 720. As described above, the brace 710 can be rotated relative to the body 720 and/or the cannula C to adjust the length L between the contact surface 712 of the brace 710 and the hub H of the cannula C. Similarly stated, the brace 710 can be rotated relative to the body 720 and/or the cannula C to adjust the length L of the retainer 700. Moreover, the openings 715 and 717 allow the retainer 700 to be disposed about the cannula C after the cannula C is disposed into the body.

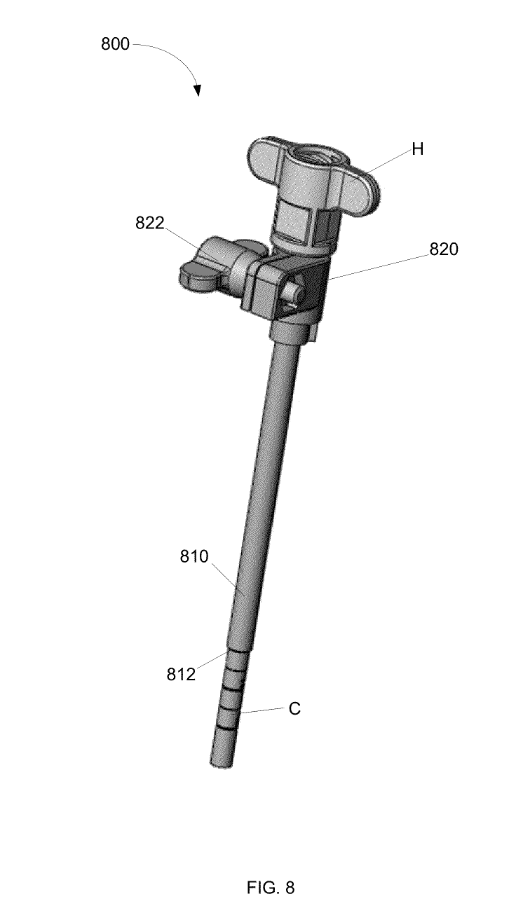

FIG. 8 illustrates a retainer 800 including a brace 810 and a body portion 820. Brace 810 includes a contact portion 812 configured to contact an internal bodily tissue, as described herein. The body portion 820 includes a clamp assembly 822 configured to maintain the cannula C in place such that it cannot move relative to the brace 810 and/or the body portion 820.

While various embodiments of the invention have been described above, it should be understood that they have been presented by way of example only, and not limitation. Where methods described above indicate certain events occurring in certain order, the ordering of certain events may be modified. Additionally, certain of the events may be performed concurrently in a parallel process when possible, as well as performed sequentially as described above.

What is claimed is:

1. An apparatus, comprising:
a first portion including a guide member and a coupling portion, the guide member and the coupling portion collectively defining a lumen configured to receive a cannula therethrough, the guide member configured to move relative to the coupling portion between a first position and a second position when the cannula is disposed within the lumen, the first portion having a first configuration when the guide member is in the first position relative to the coupling portion, the first portion having a second configuration when the guide member is in the second position relative to the coupling portion; and
a second portion coupled to a distal end of the first portion, the second portion defining a lumen configured to receive at least a portion of the cannula, the lumen of the second portion in fluid communication with the lumen of the first portion, the second portion including a contact surface configured to contact a bodily tissue of a patient when at least a portion of the cannula is disposed through the lumen of the second portion and within a body of the patient,
the second portion and the first portion collectively configured to maintain a position of the cannula relative to the bodily tissue when at least a portion of the cannula is disposed through the lumen of the first portion, the lumen of the second portion, and within the body of the patient.

2. The apparatus of claim 1, wherein the coupling portion includes threads that threadedly couples the coupling portion to the guide member.

3. The apparatus of claim 1, wherein the guide member is in the first position relative to the coupling portion during insertion of the cannula into the lumen of the second portion and into the body of the patient.

4. The apparatus of claim 1, wherein the guide member is moved from the first position to the second position relative to the coupling portion when the cannula is disposed within the body of the patient at a desired position.

5. The apparatus of claim 1, wherein the first portion has a first length when in its first configuration and a second length when in its second configuration, the second length being greater than the first length.

6. The apparatus of claim 1, wherein the contact surface of the second portion includes at least one of a flute, a knurl, or threads configured to engage a portion of the bodily tissue to limit movement of the apparatus relative to the bodily tissue.

7. The apparatus of claim 1, wherein the contact surface of the second portion is configured to contact a portion of a vertebra of the patient.

8. The apparatus of claim 1, wherein the second portion includes a joint configured to selectively adjust an angle of the first portion relative to the bodily tissue.

9. The apparatus of claim 1, wherein the first portion includes a locking mechanism configured to inhibit movement of the cannula relative to the second portion.

10. An apparatus, comprising:
a body portion having a side wall defining a lumen therethrough, the side wall having one or more threads; and
a brace portion having a side wall defining a lumen therethrough, the lumen of the brace portion fluidically coupled to the lumen of the body portion, the side wall of the brace portion having one or more threads that are configured to matingly couple to the one or more threads of the body portion,
the lumen of the body portion and the lumen of the brace portion configured to receive a cannula therethrough, the brace portion having a contact surface configured to contact a bodily tissue of a patient when the cannula is disposed within the lumen of the body portion and the lumen of the brace portion,
the brace portion and the body portion collectively configured to maintain a position of the cannula within a body of the patient relative to the bodily tissue when the cannula is disposed within the lumen of the brace portion and the lumen of the body portion.

11. The apparatus of claim 10, wherein the body portion is configured to move between a first position and a second position relative to the brace portion,
when the body portion is in the first position relative to the brace portion, the cannula is inserted within the body of the patient through the lumen of the brace portion, the body portion having a first length in the first position,
when the body portion is in the second position relative to the brace portion, the cannula is disposed within the body of the patient at a desired location, the body portion having a second length in the second position.

12. The apparatus of claim 10, wherein the body portion is configured to move between a first position and a second position relative to the brace portion,
when the body portion is in the second position relative to the brace portion, the cannula is disposed within the body of the patient and the proximal end of the body portion abuts the hub of the cannula to prevent distal movement of the cannula.

13. The apparatus of claim 10, wherein the body portion includes a locking mechanism configured to inhibit movement of the cannula relative to the brace portion.

14. The apparatus of claim 10, wherein the contact surface of the brace portion is configured to contact a vertebra of the patient,
when the contact surface contacts the vertebra, the cannula is configured to extend through an opening of the vertebra and to deliver bone cement into the vertebra.

15. The apparatus of claim 10, wherein the brace portion is configured to rotate relative to the body portion, via the one or more threads of the body portion, to adjust a length of the apparatus.

16. A method, comprising:
forming an opening in a vertebral body;
inserting a distal portion of a cannula into the opening of the vertebral body;
coupling a proximal portion of the cannula to a retainer device disposed outside the vertebral bone, the retainer device defining a lumen therethrough configured to receive the proximal portion of the cannula when the cannula is coupled to the retainer device, the retainer device having a brace portion and a body portion, the brace portion configured to contact an outer surface of the vertebral body when the proximal portion of the cannula is coupled to the retainer device, the body portion being in a first position during the coupling;
after the coupling, adjusting the retainer device about the proximal portion of the cannula to inhibit movement of the distal portion of the cannula within the vertebral body, the body portion of the retainer device being moved from the first position to the second position during the adjusting so that a proximal end of the body portion abuts a hub of the cannula.

17. The method of claim 16, wherein a distal end of the cannula includes an expandable member configured to deliver bone cement within the vertebral body.

18. The method of claim 16, wherein the inserting the distal portion of the cannula into the opening of the vertebral body occurs after the coupling the proximal portion of the cannula to the retainer device.

19. The method of claim 16, wherein the adjusting includes adjusting the retainer device about the proximal portion of the cannula such that a position of the cannula relative to the vertebral bone is maintained.

20. The method of claim 16, further comprising:
positioning the brace portion of the retainer device against the outer surface of the vertebral body prior to the coupling.
